(19) United States
(12) Patent Application Publication
(10) Pub. No.: US 2018/0169379 A1
(43) Pub. Date: Jun. 21, 2018

(54) MULTI-LUMEN MEDICAL DEVICE WITH POWDERED METAL

(71) Applicant: Cook Medical Technologies LLC, Bloomington, IN (US)

(72) Inventor: Benjaman B. Quearry, Bloomington, IN (US)

(73) Assignee: Cook Medical Technologies LLC, Bloomington, IN (US)

(21) Appl. No.: 15/838,906

(22) Filed: Dec. 12, 2017

Related U.S. Application Data
(60) Provisional application No. 62/434,852, filed on Dec. 15, 2016.

(51) Int. Cl. A61M 25/00; A61B 6/12; A61L 29/14

(52) U.S. Cl. CPC A61M 25/0023; A61M 25/0068; A61M 2025/1079; A61L 29/14; A61B 6/12

(57) ABSTRACT

A medical device may be provided for insertion into a body of a patient. The medical device includes an elongate member made of a first material. The elongate member includes a first lumen and a second lumen. The second lumen defines a chamber within the second lumen. The chamber contains powdered metal. The density of the chamber differs from the density of the first material such that the chamber is distinguishable within the elongate member under a visualization technique while the elongate member is in the body of the patient.

### CROSS-REFERENCE

This application is a continuation of U.S. Provisional Patent Application No. 62/434,852 filed Dec. 15, 2016, the specifications of which are incorporated herein in their entirety by reference.

### TECHNICAL FIELD

This disclosure relates to medical devices for use in medical procedures and, in particular, to visualization features and techniques for catheters and sheaths while inside the body of a patient.

### BACKGROUND

The statements in this section merely provide background information related to the present disclosure and may not constitute prior art.

Intraluminal medical devices such as catheters and sheaths are commonly used in veins and arteries of a patient for a variety of different purposes, such as delivery of a stent, application of a medical compound, or removal of material from a luminal passage. In these procedures, it may be desirable to visualize the medical device or a particular portion of the medical device within the body of the patient to ensure the effectiveness or accuracy of the procedure. Different visualization techniques such as radiological imaging, magnetic resonance imaging (MRI) and ultrasonic imaging may be preferable depending upon the circumstances of the procedure. The preference of visualization techniques may be dependent on the particular circumstances of the patient, the available equipment, the location of the treatment area within the patient, and other possible factors. Therefore, a medical device having a portion which can be visualized under a variety of visualization techniques is desirable.

### SUMMARY

Further areas of applicability will become apparent from the description provided herein. It should be understood that the description and specific examples are intended for purposes of illustration only and are not intended to limit the scope of the present disclosure.

In one embodiment, a medical device is provided for insertion into a body of a patient. The medical device includes an elongate member made of a first material. The elongate member comprises a first lumen and a second lumen. The second lumen defines a chamber within the second lumen. The chamber contains powdered metal. The density of the chamber differs from the density of the first material such that the chamber is distinguishable within the elongate member under a visualization technique while the elongate member is in the body of the patient.

In another embodiment, a method for using a medical device within a body of a patient is provided. The medical device includes an elongate member made of a first material. The elongate member includes a first lumen and a second lumen, where the second chamber defines a chamber within the second lumen. The method includes inserting a distal end of the elongate member into the body of the patient such that the chamber of the second lumen is within the body, and distinguishing the chamber of the second lumen from the elongate member within the body by a visualization technique. The chamber contains powdered metal where a density of the powdered metal within the chamber is different than a density of the first material.

In yet another embodiment, a method of manufacturing a medical device configured to be advanced into a body of a patient is provided. The method includes forming an elongate member having a first lumen and a second lumen. The elongate member is made from a first material. The method further includes closing the second lumen at a first location, inserting powdered metal into the second lumen, and closing the second lumen at a second location to form a chamber extending between the first location and the second location. The chamber contains the powdered metal and the density of the chamber differs from a density of the first material such that the chamber is distinguishable within the elongate member under visualization while the chamber is within the body of the patient.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates a cross-sectional side view of a first example of a medical device;

FIG. 2 illustrates a cross-sectional front view of the first example of the medical device shown in FIG. 1;

FIG. 3 illustrates a cross-sectional side view of a second example of a medical device;

FIG. 4 illustrates a cross-sectional front view of the second example of the medical device shown in FIG. 2;

FIG. 5 illustrates a cross-sectional side view of a third example of a medical device;

FIG. 6 illustrates a cross-sectional front view of the third example of the medical device shown in FIG. 5;

FIG. 7 illustrates a cross-sectional side view of a lumen of a fourth example of a medical device;

FIG. 8 illustrates a flow diagram of operations to use a medical device within a body of a patient; and

FIG. 9 illustrates a flow diagram of operations to manufacture a medical device.

The drawings described herein are for illustration purposes only and are not intended to limit the scope of the present disclosure in anyway.

### DETAILED DESCRIPTION

The following description is merely exemplary in nature and is not intended to limit the present disclosure, application, or uses.

In one example, a medical device is provided for insertion into a body of a patient. The medical device includes an elongate body made from a first material. The elongate member comprises a first lumen and a second lumen. The second lumen defines a chamber within the second lumen. The chamber contains powdered metal. In one example, the density of the chamber differs from the density of the first material such that the chamber is distinguishable within the elongate member under a visualization technique while the elongate member is in the body of the patient.

One technical advantage of the systems and methods described below may be that the medical device or a particular portion of the medical device may be located and observed under visualization while in the body. Another technical advantage may be that the same medical device may be under a variety of visualization techniques.

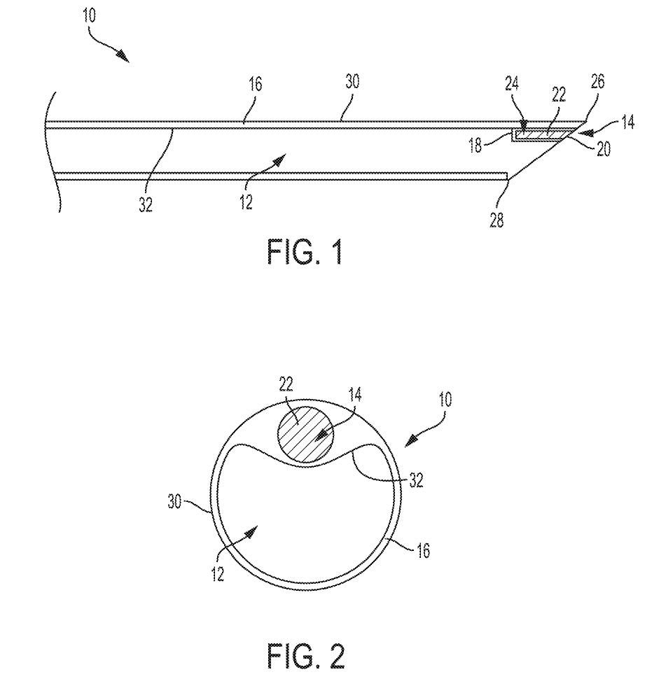

FIG. 1 illustrates a cross-sectional side view of a medical device having an elongate member 10. The elongate member 10 may be any object which is configured to extend intraluminally within a body of a patient. Examples of the elongate member 10 may include a catheter, a sheath, a wire guide, and a needle. The elongate member 10 may include an outer surface 30 and an inner surface 32. The elongate member 10 may be made of polymers such as polyvinylchloride, polyurethane, and silicone or metals such as stainless steel, titanium, monel, and inconel. For example, and as illustrated in FIG. 1, the elongate member 10 may be a needle having a sharpened distal tip (26, 28). The sharpened distal tip (26, 28) may extend from a heel 28 to a point 26 at the distal end. The elongate member 10 may have an opening 11 at the sharpened distal tip (26, 28).

The elongate member 10 shown in FIG. 1 includes a first lumen 12 and a second lumen 14. The first lumen 12 and the second lumen 14 may be any space in the interior of the elongate member 10. Examples of the first lumen 12 and the second lumen 14 may include passageways, tubes, and other volumes within the elongate member 10. The first lumen 12 and the second lumen 14 may extend along the entire length of the elongate member 10 or may occupy only a portion of the length of the elongate member 10. The first lumen 12 and the second lumen 14 may have any cross-sectional shape, such as circular, semi-hemispheric, crescent shaped, or any other suitable shape. As illustrated in the example provided in FIG. 1, the first lumen 12 may extend along the entire length of the elongate member 10 and may be adapted to allow passage of medical devices such as stents, guide wires, and other devices (not shown) through the first lumen 12.

Also illustrated in FIG. 1, the second lumen 14 may occupy only a portion of the length of the elongate member 10. The second lumen 14 may be closed at a first location 18 and a second location 20 to define a chamber 24 between the first location 18 and the second location 20. The chamber 24 may be any portion of the second lumen which may be adapted to contain powdered material. Examples of the chamber 24 may include a segment, a pocket, and a tube. The chamber 24 may extend the entire length of the second lumen 14 or may be located only within a portion of the second lumen 14. The chamber 24 may contain powdered metal 22. The chamber 24 may be sealed at the first location 18 and the second location 20 to prevent the powdered metal 22 or other material from exiting the chamber 24.

The powdered metal 22 may be any metallic substance which has the consistency of fine particles. The particles of the powdered metal 22 may be chemically and mechanically uncoupled to one another. The individual particles of the powdered metal 22 may vary in diameter between 3 micrometers and 1 millimeter. Examples of the powdered metal 22 may include tungsten, nickel, and other minimally bio-reactive metals. In some embodiments, particularly where visualization using magnetic resonance imaging is likely to be used, the powdered metal 22 may be non-ferrous. In order to facilitate visualization, the powdered metal 22 may have a density which is different from the material of the elongate member 10. From this different density, the chamber 24 may also have a density which is different from the material of the elongate member 10. For example, a powdered metal 22 which has a density which is higher than the material of the elongate member 10 may allow the chamber 24 to have a higher density than the material of the elongate member 10 and may be visible within the body of the patient under a variety of visualization techniques. The density of the powdered metal 22 may be between 8.9 grams per cubic centimeter and 20 grams per cubic centimeter. The density of the chamber 24 may be between 8.0 grams per cubic centimeter and 20 grams per cubic centimeter. Where the elongate member 10 may be a metallic needle, the density of the material of the elongate member 10 may be between 4.5 grams per cubic centimeter and 7.9 grams per cubic centimeter. Where the elongate member 10 may be a polymer catheter, the density of the elongate member 10 may be between 1 gram per cubic centimeter and 2.5 grams per cubic centimeter. Furthermore, the powdered metal 22 may have a density which is different from the body of the patient and any intraluminal fluid. For example, a powdered metal 22 which has a density which is higher than the luminal walls of the patient and any intraluminal fluid may allow the chamber 24 and the powdered metal 22 to be visible within the body of the patient under a variety of visualization techniques. For example, typical densities of luminal walls of patients may be between 0.9 grams per cubic centimeter and 1.08 grams per cubic centimeter. Typical densities of intraluminal fluids, such as blood or saline, may be between 1.02 grams per cubic centimeter and 1.2 grams per cubic centimeter.

As illustrated in FIG. 1, where the elongate member 10 is a needle, the second lumen 14 may be located at or near the sharpened distal tip (26, 28) of the elongate member 10. The chamber 24 of the second lumen 14 may be sealed or closed at the first location 18 which is located at the heel 28 of the elongate member 10. The chamber 24 may be sealed or closed at the second location 20 which is located at the point 26 of the elongate member 10. Such a configuration may allow the longitudinal position of the sharpened distal tip (26, 28) of the elongate member 10 to be visible and distinguishable from the rest of the elongate member 10 while in the body using a visualization technique.

FIG. 2 illustrates a cross-sectional front view of the medical device shown in FIG. 1. The first lumen 12 and the second lumen 14 may be arranged in a side-by-side fashion, wherein the first lumen 12 extends longitudinally along a first side of the elongate member 10 and the second lumen 14 extends along a second side of the elongate member 10. The first lumen 12 may be larger than the second lumen 14 such that the first lumen 12 may comprise between 40% and 80% of a cross-sectional area of the elongate member 10. Comparatively, the second lumen may between 10% and 40% of a cross-sectional area of the elongate member 10. In some embodiments, as illustrated in FIG. 1, the second lumen 14 may only overlap with a portion of the first lumen 12. Where the second lumen 14 does not overlap with the first lumen 12, the first lumen 12 may comprises a larger portion of the cross-sectional area of the elongate member 10.

In some embodiments, the second lumen 14 may be embedded within a wall 16 of the elongate member 10. In such configurations, the inner surface 32 of the elongate member 10 may define the first lumen 12. In such configurations, though the second lumen 14 may be embedded within the wall 16 of the elongate member 10, the second lumen 14 and the chamber 24 may be materially distinct from the wall 16 such that the material of the elongate member 10 does not mix with the powdered metal 22 contained in the chamber 24. To accomplish this, the second lumen 14 may be defined by a liner (44 in FIG. 6) which surrounds the second lumen 14 and separates the second lumen 14 from the wall 16 of the elongate member 10.

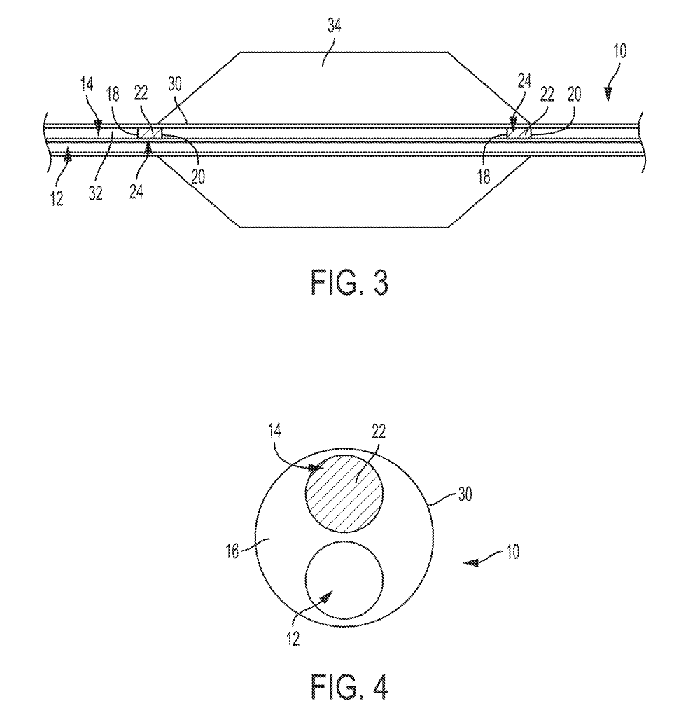

FIGS. 3 and 4 illustrate cross-sectional views of another embodiment of the medical device. The elongate member 10 may include a balloon 34 extending longitudinally along an outer surface 30 of the elongate member 10. The balloon may be inflatable to expand laterally and outward from the outer surface 30 of the elongate member 10. The first lumen 12 may be configured to delivery inflation fluid to an interior of the balloon 34 to inflate the balloon 34.

The second lumen 14 may be adapted to indicate the location of the balloon 34 using a visualization technique. The chamber 24 containing powdered metal 22 may extend longitudinally from a proximal end 46 of the balloon 34 to a distal end 48 of the balloon. Alternatively, the second lumen 14 may include a plurality of chambers 24 containing powdered metal 22. In such a configuration, each of the plurality of chambers 24 would be spaced apart from each other to indicate a specific location on the elongate member 10 under visualization. For example, as illustrated in FIG. 3, a chamber 24 may be located at the proximal end 46 of the balloon 34. An additional chamber 24 may be located at the distal end 48 of the balloon. With such a configuration, the location of the balloon 34 may be readily apparent using a visualization technique. The portions of second lumen 14 which do not contain the chambers may be filled with a variety of materials, such as polymers, saline, non-powdered metals or other suitable materials which would provide a visual contrast with the chambers 24 when using a visualization technique.

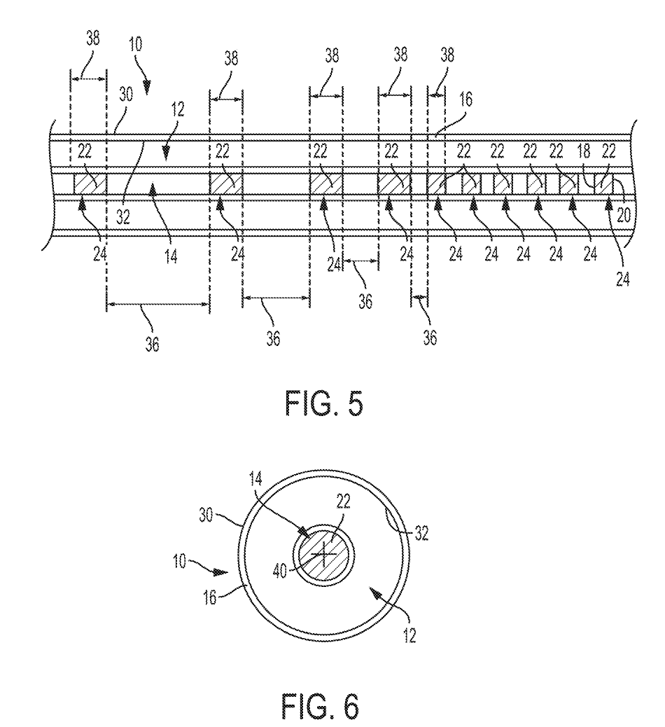

FIGS. 5 and 6 illustrate cross-sectional views of yet another embodiment of the medical device. The elongate member 10 may have a first lumen 12 and a second lumen 14 which are concentric to a longitudinal axis 40 at the center of the cross-section of the elongate member 10. As illustrated in FIG. 6, the second lumen 14 may be arranged within the first lumen 12. Such a configuration may be ideal when the first lumen 12 is adapted to provide a fluid through the elongate member 10. Alternatively, the first lumen 12 may be arranged within the second lumen 14. In such a configuration, the second lumen 14 may have a smaller cross-sectional area and may be arranged as a thin shell around a larger central first lumen 12. Such a configuration may be ideal when the first lumen 12 is adapted to provide access for a medical device such as a stent or wire guide.

As illustrated in FIG. 5, the second lumen 14 may include a plurality of chambers 24 containing powdered metal 22 spaced apart from each other at regular intervals. The width 38 of the chambers 24 and the length 36 between chambers 24 may be consistent along a portion of or the entire length of the second lumen 14. Alternatively, the width 38 of the chambers 24 and the length 36 between the chambers 24 may vary along the length of the second lumen 14. Such a configuration may be useful in providing a visual basis for making measurements within the body of the patient using a visualization technique. For example, the width 38 of the chambers 24 and the length 36 between the chambers 24 may be narrow close to the distal end (26, 28) of the elongate member 10, where accurate measurements of the patient's physiology and the precise location of a supplementary device may be particularly useful. However, the width 38 of the chambers 24 and the length 36 between the chambers 24 may lengthen as the second lumen 14 extends proximally, and as smaller incremental measurements become less necessary. The width 38 of the chambers may be between 0.25 millimeters and 1 millimeter. The length between the chambers may be between 0.25 millimeters and 10 millimeters. The portions of the second lumen 14 between the chambers 24 may be filled with stainless steel or another material which provides a visual contrast with the powdered metal 22 in the chambers 24 while using a visualization technique.

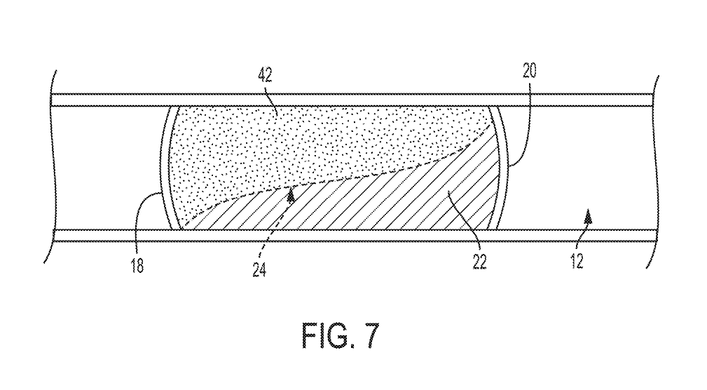

FIG. 7 illustrates a cross-sectional view of the second lumen 14 of an embodiment of the elongate member 10. As illustrated, the chamber 24 may be partially filled with powdered metal 22 and partially filled with a filler material 42. The filler material 42 may be any material which, when combined with the powdered metal, allows the chamber 24 to be visually distinct from the material of the elongate member 10 while using a visualization technique. Examples of the filler material 42 may include air, saline, silicone, a polymer, or any suitable substance. The filler material 42 may be a liquid, gas, or suspension which allows the powdered metal 22 to retain its particulate nature and allow the powdered metal 22 to move throughout the chamber 24. Alternatively, the filler material 42 may be a solid or semi-solid material which occupies a portion of the chamber 24 and prevents the powdered metal 22 from occupying an overlapping portion of the chamber 24. The powdered metal 22 may comprise between 1% and 100% of the volume of the chamber.

In some embodiments, the filler material 42 may be mixed in with the powdered metal 22 such that the powdered metal is maintained in a virtual solution. A ratio of powdered metal 22 to filler material 42 in the virtual solution may be controlled to prevent harmonic vibrations associated with metallic devices used in an MRI suite.

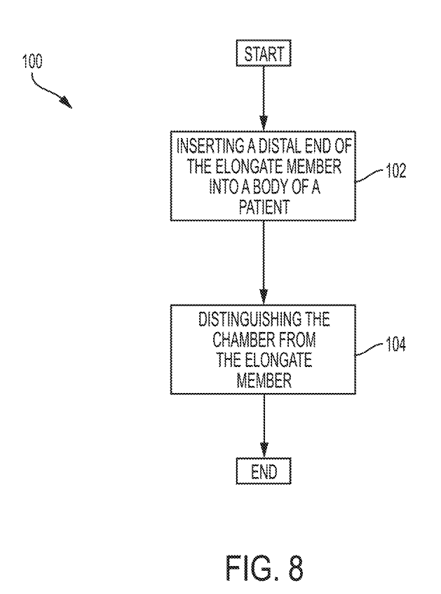

FIG. 8 illustrates a flow diagram of operations to use the medical device. The operations (100) may include fewer, additional, or different operations than illustrated in FIG. 8. Alternatively or in addition, the operations (100) may be performed in a different order than illustrated.

The distal end (26, 28) of the elongate member 10 may be inserted into the body of the patient (102). While inserting the elongate member 10, the chamber 24 containing powdered metal 22 may be inserting into the body of the patient as well. Once the chamber 24 containing the powdered metal has been advanced to the target destination, a visualization technique may be utilized to distinguish the chamber 24 of the second lumen 14 from the elongate member 10 within the body of the patient (104). The chamber 24 may be distinguishable from the material of the elongate member 10 because the chamber 24 contains powdered metal 22, raising the density of the chamber 24 such that it has a different density than the material of the elongate member 10. The visualization technique may be any technique which is used to observe the location, condition, or arrangement of an intraluminal passage or objects within an intraluminal passage. Examples of the visualization technique may be ultrasonic visualization, radioscopic imaging, and magnetic resonance imaging, as well as other techniques.

In some embodiments, the chamber 24 of the second lumen 14 is located at a particular longitudinal portion in the elongate member 10 and may not extend proximally or distally from that longitudinal portion. In such embodiments, the longitudinal position of a portion of the elongate member 10 may be determined visually while distinguishing the chamber 24 from the material of the elongate body 10, where the portion of the longitudinal elongate member 10 is in the vicinity of the chamber 24. Where the chamber 24 is predetermined at a known longitudinal position within the elongate member 10, observation of the chamber 24 may assist in determining the longitudinal position of the elongate member 10.

In some embodiments, the chamber 24 of the second lumen 14 is laterally offset from the longitudinal axis 40. In such configurations, the rotational orientation of the elongate member 10 may be determine while distinguishing the chamber 24 from the material of the elongate body 10. If the walls 16 or other features of the elongate body 10 are visible using a visualization technique, a predetermined lateral offset of the chamber may be used to compare with the walls 16 to determine the rotational orientation of the elongate member 10.

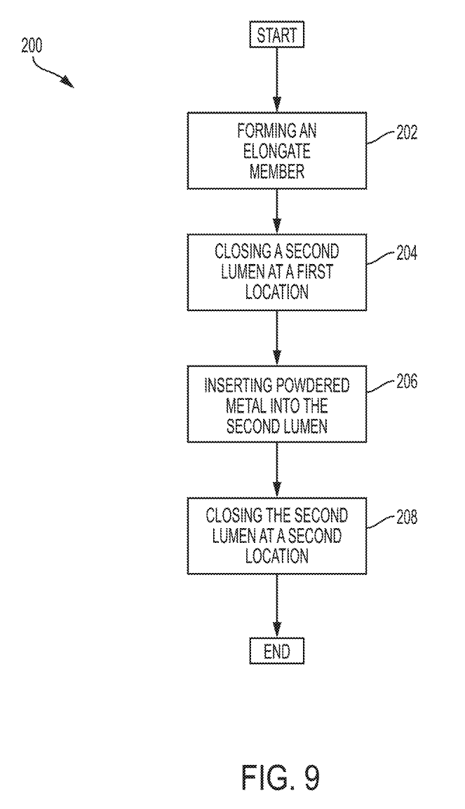

FIG. 9 illustrates a flow diagram of operations to manufacture the medical device. The operations (200) may include fewer, additional, or different operations than illustrated in FIG. 9. Alternatively or in addition, the operations (200) may be performed in a different order than illustrated.

The medical device may be formed in a manufacturing process. Initially, an elongate member 10 is formed (202). The elongate member 10 may be formed with a first lumen 12 and a second lumen 14. The first lumen 12 and the second lumen 14 may be formed while forming the elongate member 10 using mandrels or may be created within the elongate member 10 after forming the elongate member 10. After the second lumen 14 is formed, the second lumen 14 is closed or sealed at a first location 18 (204). After the second lumen 14 is closed at the first location 18 (204), the second lumen 14 may filled with powdered metal 22 (206). After the powdered metal 22 has been inserted into the second lumen 14, the second lumen 14 may be closed or sealed at a second location 20 (208). The powdered metal 22 is contained within a chamber 24 defined by being between the first location 18 and the second location 20. A filler material 42 may also be inserted into the second lumen before the second lumen 14 is sealed at the second location 20.

In some embodiments, the operations (200) may include forming the second lumen 14 independently of the elongate member 10. The second lumen may be formed by a liner 44 which encircles the second lumen 14. The liner 44 defining the second lumen 14 may be inserted into the elongate member 10 and bonded to the inner surface 32 of the elongate member 10. Alternatively, the second lumen 14 may be embedded within a wall 16 of the elongate member 10 as the elongate member 10 is being formed.

In addition to the advantages that have been described, it is also possible that there are still other advantages that are not currently recognized but which may become apparent at a later time. While various embodiments have been described, it will be apparent to those of ordinary skill in the art that many more embodiments and implementations are possible. Accordingly, the embodiments described herein are examples, not the only possible embodiments and implementations.

What is claimed is:

1. A medical device for insertion into a body of a patient, comprising,
an elongate member being made of a first material and comprising a first lumen and a second lumen, the second lumen defining a chamber within the second lumen, the chamber containing powdered metal, wherein a density of the chamber differs from a density of the first material such that the chamber is distinguishable within the elongate member under a visualization technique while the elongate member is in the body of the patient.

2. The medical device of claim 1, wherein the second lumen is arranged within the first lumen.

3. The medical device of claim 1, wherein the first lumen extends longitudinally along a first side of the elongate member and the second lumen extends along a second side of elongate member.

4. The medical device of claim 1, wherein the second lumen has a smaller cross-sectional area than the first lumen and wherein the second lumen is offset from a center of a cross-section of the elongate member.

5. The medical device of claim 4, wherein the cross-sectional area of the second lumen is less than 40% of a cross-sectional area of the elongate member.

6. The medical device of claim 1, wherein the second lumen comprises a plurality of chambers, each of the plurality of chambers containing powdered metal and each of the plurality of chambers being longitudinally spaced apart from each other.

7. The medical device of claim 1, wherein the elongate member comprises a balloon extending along an outer surface of the elongate member, and wherein the second lumen comprises a chamber containing powdered metal longitudinally aligned with one of a first end of the balloon and a second end of the balloon.

8. The medical device of claim 1, wherein the elongate member comprises a needle having a distal tip, wherein the chamber of the second lumen is located at the distal tip of the needle.

9. The medical device of claim 1, wherein the elongate member is a polymer catheter.

10. A method of using a medical device within a body of a patient, the medical device comprising an elongate member made of a first material, the elongate member comprising a first lumen and a second lumen, the second lumen defining a chamber within the second lumen, the method comprising,
inserting a distal end of the elongate member into the body of the patient such that the chamber of the second lumen is within the body; and
distinguishing the chamber of the second lumen from the elongate member within the body by use of a visualization technique, wherein the chamber contains powdered metal, a density of the powdered metal within the chamber being different from a density of the first material.

11. The method of claim 10, further comprising determining a longitudinal position of a portion of the elongate member within the body, the portion being in a vicinity of the chamber of the second lumen.

12. The method of claim 10, determining a rotational orientation of the elongate member within the body by comparing a position of the chamber of the second lumen relative to the elongate member, wherein the second lumen is offset from a center of a cross-section of the elongate member.

13. The method of claim 10, wherein the visualization technique is ultrasound.

14. The method of claim 10, wherein the visualization technique is magnetic resonance imaging.

15. A method of manufacturing a medical device configured to be advanced into a body of a patient, the method comprising,
forming an elongate member having a first lumen and a second lumen, the elongate member being made of a first material;
closing the second lumen at a first location;
inserting powdered metal into the second lumen; and
closing the second lumen at a second location to form a chamber extending between the first location and the second location, the chamber containing the powdered metal, wherein a density of the powdered metal within the chamber differs from a density of the first material such that the chamber is distinguishable within the elongate member under visualization while the chamber is within the body of the patient.

16. The method of claim 15, further comprising inserting a filler material into the chamber.

17. The method of claim 16, wherein the powdered metal fills at least 50% of a volume of the chamber.

18. The method of claim 16, wherein the filler material is saline.

19. The method of claim 15, wherein forming the elongate member comprises bonding the second lumen to an inner surface of the elongate member.

20. The method of claim 15, wherein forming the elongate member comprises embedding the second lumen into a wall of the elongate member.
